(19) United States
(12) Patent Application Publication
(10) Pub. No.: US 2025/0380855 A1
(43) Pub. Date: Dec. 18, 2025

(54) CHIP-IN-TIP ENDOSCOPE WITH IMPROVED 3D VISION

(71) Applicant: Schölly Fiberoptic GmbH, Denzlingen (DE)

(72) Inventors: Cyril AUTOURDE, Kunheim (FR); Hannes JOSEPH, Bahlingen (DE); David HENSLE, Freiburg (DE)

(73) Assignee: Schölly Fiberoptic GmbH, Denzlingen (DE)

(21) Appl. No.: 19/234,929

(22) Filed: Jun. 11, 2025

(30) Foreign Application Priority Data
Jun. 12, 2024 (DE) 102024116457.3

(51) Int. Cl. A61B 1/00; A61B 1/05; G02B 27/10

(52) U.S. Cl. CPC A61B 1/00193; A61B 1/00057; A61B 1/00194; A61B 1/05; G02B 27/1013

(57) ABSTRACT

To improve the image quality of stereoscopic images which are recorded using a CIT endoscope (1), it is provided that an optical correction element (11), which shifts respective main beams (20a, 20b) of the respective optical channel (4a, 4b) axially parallel so that imaging beam paths (5a, 5b), which are generated by the two optical channels (4a, 4b) approach one another, is arranged between two optical channels (4a, 4b), which are used for imaging, extend parallel and are formed identically, and the image sensor (2) used for imaging. As a result, an image center distance D between image areas (6a, 6b) on a sensor surface (7) of the image sensor (2) that is used for both individual images can thus be reduced in comparison to an entry-side stereo base B defined by the two optical channels (4a, 4b), so that a higher image resolution can be achieved.

### CROSS-REFERENCE TO RELATED APPLICATIONS

This application claims priority from German Patent Application No. 10 2024 116 457.3, filed Jun. 12, 2024, which is incorporated herein by reference as if fully set forth.

### TECHNICAL FIELD

The invention relates to a stereoscopic chip-in-tip (CIT) endoscope, which has a distal image sensor arranged in a distal tip segment of the endoscope, and furthermore two optical channels, which each form an imaging beam path (in particular by means of optical lenses and possibly further optical elements). Each imaging beam path generates a respective image area used for the imaging in this case: The left channel illuminates a left image area and the right channel illuminates a corresponding right image area on a sensor surface of the image sensor. Areas of pixels which are in the form of a circular disk or are rectangular can be read out within these image areas on the sensor surface in order to obtain respective individual images (of the left or the right channel). Respective geometrical image center points of these two image areas define an image center distance D (on the sensor surface) in this case. Furthermore, each of the two imaging beam paths defines an associated respective optical axis which extends through the respective optical channel. The two optical axes are arranged here on the entry side at a distance B, which defines a stereo base of the endoscope. This endoscope is therefore configured for stereoscopic vision or for recording stereoscopic images, so that 3D imaging or 3D visualization is possible using the endoscope.

### BACKGROUND

Such CIT endoscopes have been previously known per se for some time in the prior art and are used in particular to offer a perspective view to the operation area, therefore a three-dimensional impression, to the physician during surgical procedures inside the body, which can significantly facilitate the operation, more precisely the manual control of the operation instruments. The three-dimensional impression which results in the brain of the user depends here on the size of the mentioned stereo base. The stereo base is in this case the distance of the two optical axes, using which the endoscope looks at the object to be observed or via which it receives imaging light beams from this object.

### SUMMARY

The invention is based on the object of enabling improved 3D imaging using a CIT endoscope. In particular, the quality of the stereoscopic images recorded using the endoscope is to be improved.

According to the invention, one or more of the features disclosed herein are provided in an endoscope to achieve this object. In particular, it is therefore provided according to the invention to achieve the object in a CIT endoscope of the type mentioned at the outset that the endoscope comprises an optical correction element, which (with respect to the respective imaging beam path) is arranged between the two optical channels and the at least one image sensor. The optical correction element is designed here so that at least one, but preferably both, of the above-mentioned two optical axes (which are predetermined by the respective optical channel) shifts, preferably axially parallel, so that D<B applies. That is to say that the resulting image center distance D is less than the stereo base.

Depending on the image sensor used, in particular if it does not have high requirements for the so-called chief ray angle (CRA), a non-axially parallel shift is also possible. In this case, the respective optical axis can also be incident on the image sensor surface at a (slightly) inclined angle. An axially parallel shift is preferred, however, because then in particular it can be achieved that the respective optical axis, after the shift in the correction element, can be incident in each case perpendicularly on the sensor surface of the image sensor. A “matching”/a correspondence of the CRA between imaging optical unit and image sensor can thus be achieved, which is advantageous to avoid shadows and color errors. Lateral chromatic aberrations upon the passage through the correction element can also be limited better with axially parallel shift.

In other words, the optical correction element therefore reduces the distance between the two optical axes. In this way, in particular it is achieved that the two image areas on the active sensor surface of the image sensor advance closer to one another. This has the technical effect that the active sensor surface of the image sensor can be utilized better for the imaging in the sense that more pixels are available for the respective individual image. The image resolution can therefore be increased for each of the two individual images, which are sensorially captured within the respective image area, and therefore stereoscopic images of improved resolution can be obtained overall.

An optical correction element in the meaning of the invention can be assembled here from multiple optical components, as will become clearer on the basis of examples.

The two optical channels therefore comprise a left and a right optical channel. In this case, the left optical channel generates a left imaging beam path, which defines the left image area, and the right optical channel generates a right imaging beam path, which defines the right image area.

The resulting (active) sensor surface of the image sensor will typically be larger here than the added surfaces of the two image areas, which are actually utilized for imaging. This applies in particular if, for example, circular image areas are used (as is typical in stereo endoscopes which are used for minimally invasive surgery) or if (which is advantageous for several reasons) a narrow unused area of the sensor surfaces remains between the left and the right image area, which is respectively used for imaging and remains unconsidered in the imaging.

The use of such an optical correction element can in particular cause decentering of the respective optical axis. Preferably, both optical axes can be shifted in parallel toward one another in this case by the optical correction element (which can consist of multiple parts) and can thus be moved toward one another. In other words, the two optical axes can therefore have a greater distance upon entry into the optical correction element than upon their exit from same.

The optical correction element can therefore deflect at least one of the two optical axes so that the image center distance D is reduced, in comparison to a situation in which no such deflection element is used in the endoscope. In this case, the optical correction element can decenter at least one, but preferably both, of the two optical axes, in particular such that the two image center points advance to one another.

In classic stereo endoscopes, the respective optical axis along the respective left optical channel does not experience a deflection such that its distance to the optical axis of the adjacent right channel is changed. Therefore, in these previously known stereoscopic and endoscopic imaging systems, the distance between the image center points of the two individual images corresponds to the entry-side stereo base, which is defined on the entry side of the two optical channels by the distances between the first lenses of the imaging optical unit. The larger the resulting stereo base, the larger the three-dimensional impression here, which an observer receives upon observing the stereo images, which is typically advantageous to enable a good 3D perception. With a large stereo base, however, valuable sensor surface of the image sensor is often not utilized for imaging in previously known systems, which has a negative effect on the achievable image resolution.

The image-side/entry-side distance between the two optical axes at a distal end of the two optical channels, which defines the stereo base B, therefore establishes a three-dimensional impression of stereoscopic imaging achievable using the endoscope. It is provided according to the invention that the image center distance D deviates from the stereo base B, in particular results as less than the stereo base B, due to the optical deflection which the optical correction element causes.

If only a limited wavelength spectrum is used for imaging, it is thus technically possible to form the optical correction element by means of diffractive optical elements which cause the desired beam deflection.

In addition, it is also possible to form an optical correction element according to the invention (at least partially) by means of reflective optical elements such as, for example, inclined planar mirror surfaces.

According to the invention, the object can also be achieved by further advantageous embodiments which are explained in more detail hereinafter and recited in the claims.

For example, it is advantageous if a respective entire imaging optical unit of the respective optical channel is arranged before the optical correction element in the direction of the respective imaging beam path. This is because in this way the occurrence of image errors can be avoided in comparison to the placement of the optical correction element within the respective optical channel.

It can therefore be provided in particular that the image sensor directly follows the optical correction element, so that imaging light beams emerging from the optical correction element are incident directly on the sensor surface without passing through a further optical element, with the exception of a cover glass which protects the sensor surface of the image sensor.

One particular embodiment provides that the optical correction element has an entry surface and two exit surfaces spatially separated from one another. In this case, each of the two imaging beam paths (of the left and right optical channel) is thus split by the correction element, so that each of the two imaging beam paths exits from each of the two exit surfaces of the correction element. It is preferred in this case when the correction element has a wavelength-selective mirror surface, which guides wavelengths in a first wavelength range onto a first exit surface of the correction element and wavelengths in a second wavelength range, which deviates from the first, onto a second exit surface of the correction element. This is because in this way one image can be sensorially acquired in each of the two different wavelength ranges for each of the two imaging beam paths which generate the two optical channels. In this way, two stereoscopic images can therefore be captured in the two different wavelength ranges using the stereoscopic endoscope.

An embodiment which is particularly suitable for this purpose provides that each of the above-mentioned entry or exit surfaces of the optical correction element is formed by a respective double glass wedge, wherein this double glass wedge can preferably be formed symmetrically. The respective double glass wedge forms optical surfaces here which are inclined in relation to one another and are each formed planar. Depending on the placement of the double glass wedge on the entry side or on the exit side, the optical surfaces can be inclined differently. In such a design, the respective imaging beam path can then extend through only one of these two optical surfaces in each case.

Such an embodiment of the optical correction element is particularly suitable if the endoscope has two spatial separated image sensors, which are each configured for imaging in different wavelength ranges. In this case, a first stereoscopic imaging beam path, which ends in the first of the two image sensors, can exit through the first exit surface of the optical correction element, and a second stereoscopic imaging beam path, which ends in the second of the two image sensors, can exit through the second exit surface of the optical correction element. Each of these two stereoscopic imaging beam paths can define a separate image center distance D1 or D2 on the sensor surface of the respective image sensor here. And furthermore, both of these resulting image center distances D1, D2 can be less than the stereo base B with the aid of the optical correction element.

The first wavelength range can comprise, for example, the visible spectrum (VIS). And the second wavelength range can comprise wavelengths outside the visible spectrum, in particular in the near infrared (NIR).

As already mentioned above, the optical correction element can comprise a wavelength-selective beam splitter. In this case, two spatially separated image sensors can be assigned to the beam splitter. In this case, respective imaging in two different wavelength ranges using the two image sensors is enabled, as already explained above. In such an embodiment, it is preferred if the beam splitter spatially separates imaging beams of the respective imaging beam path (which the two optical channels generate) into a left first imaging path and a right first imaging path and into a left second imaging path and a right second imaging path. This is because in this way two stereoscopic images can be sensorially captured in different wavelength ranges.

In an embodiment having two imaging paths (each designed stereoscopically), it can be provided that the optical correction element shifts both the location of at least one optical axis of the two first imaging paths and the location of at least one optical axis of the two second imaging paths. This can be designed in particular such that both a first image center distance D1 associated with the first imaging paths and a second image center distance D2 associated with the second imaging paths is/has been adapted, preferably reduced, by the correction element.

If such a beam splitter is used and the left and right imaging beam paths are used, there are therefore four optical paths: The left and right first imaging path, which both end on the first image sensor, and the right and left second imaging path, which both end on the second image sensor. The beam splitter can be designed in one piece here, in particular by means of a prism having wavelength-selective mirror surface, or else can be assembled from multiple optical components (for example, from two such prisms, thus in each case one prism per optical channel of the endoscope).

The splitting into the two first imaging paths and the two second imaging paths can take place here with the aid of a wavelength-selective mirror surface of the beam splitter. A glass body can join this mirror surface, in particular to thus enable compensation of the optical path length differences between the first and second imaging paths.

Each of the two optical channels of the endoscope that are mentioned at the outset can have a respective imaging optical unit, which comprises an objective optical unit and a downstream relay optical unit. The relay optical unit can image an image from an intermediate image plane, which is generated by the objective optical unit, through the optical correction element on the sensor surface of the at least one image sensor. If the optical correction element has a beam splitter in this case, the relay optical unit will thus image in each case one image on the respective sensor surface of the respective assigned image sensor through the optical correction element in a wavelength-dependent manner.

Preferably, the respective imaging optical unit can additionally comprise a deflection prism arranged before the objective optical unit and a concave lens arranged before the deflection prism. The endoscope can thus be designed having an oblique view.

It is particularly preferred if the optical correction element is formed by means of at least two optical deflection elements. The deflection elements can be formed identically and/or complementarily to one another here. Furthermore, the deflection elements can in particular be implemented by means of complementary glass wedges. This is because by way of such designs, it is achievable that the two optical deflection elements interact so that the respective associated optical axis is shifted in parallel after passage through the optical correction element. This can be designed in particular such that the two optical axes extend at reduced distance (after traversing the optical correction element) and the left and the right image area therefore approach one another on the sensor surface.

“Complementary” can be understood technically here to mean that the two deflection elements are designed so that the respective effected beam deflections thereof mutually compensate so that the respective optical axis is shifted in parallel after passage through the optical correction element. Depending on the embodiment, the deflection elements can also be formed by means of refractive or diffractive elements, wherein combinations of such elements (also having refractive elements) can also be formed.

The at least two optical deflection elements can preferably be applied to an entry surface and to an exit surface of the beam splitter. In this case, it is furthermore preferred if a third optical deflection element is applied to an exit surface of a glass body, wherein this glass body is placed on a wavelength-selective mirror surface of the beam splitter. This wavelength-selective mirror surface forms a first exit surface of the beam splitter here. The second optical deflection element can in this case be applied to a second exit surface of the beam splitter.

In all of the above-mentioned embodiments, an entry surface and an associated exit surface of the optical correction element can be aligned parallel to one another. In this case, however, the entry surface and the exit surface of the optical correction element can each extend differently in relation to the sensor surface of the at least one image sensor, namely either parallel or else nonparallel. In both cases, the desired shift of the respective optical axis can be achieved.

Furthermore, in all of the above-mentioned embodiments, the respective main beam of the respective imaging beam path can extend obliquely in relation to a longitudinal axis of the endoscope during the passage through the optical correction element and parallel to the longitudinal axis after exit from the optical correction element.

According to one embodiment, a respective main beam (of the respective imaging beam path) can be deflected at least four times (thus in particular refracted at least four times) during the passage through the optical correction element. For example, the respective main beam, during the passage through the optical correction element, can initially experience a first optical refraction at an air/glass interface, then a second optical refraction at a glass/glass interface, then a third optical refraction at a glass/glass interface, and finally a fourth and last optical refraction at a glass/air interface. In this way, the respective main beam can overall be shifted axially parallel.

In contrast, a somewhat different embodiment provides that the respective main beam, during the passage through the optical correction element, is refracted twice at an internal glass/glass interface and in this case the entry surface and the exit surface of the optical correction element extend parallel to the sensor surface of the at least one image sensor. In this case, the main beam is not refracted upon entry into the optical correction element but also in particular upon exit from same. For further imaging beams which are incident obliquely on the entry or exit surface of the optical correction element, however, a respective deflection can take place there.

According to the invention, the following value ranges can be selected for the ratio of stereo base B and the center distance D:D/B<0.95, preferably even D/B<0.90 can apply.

As mentioned above, the optical correction element can be implemented as a refractive optical element, in particular by means of at least two glass wedges. In this case, it is preferred if the at least two glass wedges jointly refractively deflect at least one, but preferably both, of the optical axes so that the respective axis is shifted in parallel.

Furthermore, it is preferred for optimum imaging if an entry surface of a first glass wedge of the at least two glass wedges and an associated exit surface of a second glass wedge of the at least two glass wedges are aligned parallel to one another.

An interior angle of the respective glass wedge can preferably be less than 5° here, in particular less than 3.5°.

A normal of a first entry surface of the optical correction element, thus in particular a normal of a first entry surface of one of the glass wedges, can be aligned either axially parallel or else obliquely to the associated optical axis. In both cases, a desired axially parallel shift of the respective optical axis can be achieved. Therefore, a normal of a last exit surface of the optical deflection element, thus in particular a normal of a last exit surface of one of the glass wedges, can likewise also either be aligned axially parallel or else obliquely to the associated optical axis.

According to the invention, the respective deflection, caused by the optical correction element, of at least one or even both of the two optical axes can be designed such that the two image areas do not (yet) overlap on the sensor surface. In this case, a safety area therefore remains between the two image areas, which is not used for imaging. However, this can be made comparatively narrow. According to the invention, widths of at least 50 μm are expedient for this purpose in order to still be able to compensate for manufacturing-related variations.

If measures are sufficiently taken to minimize scattered light and the mechanical tolerances during assembly are sufficiently controlled, it is thus also possible to design the respective deflection, caused by the optical correction element, of at least one or even both of the two optical axes such that the two image areas overlap in an overlap area on the sensor surface. Light from the left and the right channel therefore gets into this overlap area. In such a case, however, respectively smaller image subareas, which are each located within one of the two image areas, can then also be used for imaging. These image subareas can preferably be selected here as rectangular, so that then rectangular individual images of high resolution can be obtained.

### BRIEF DESCRIPTION OF THE DRAWINGS

The invention will now be described in more detail on the basis of exemplary embodiments, but is not restricted to these exemplary embodiments. Further designs of the invention can be obtained from the following description of a preferred exemplary embodiment in conjunction with the general description, the claims, and the drawings. In the following description of various preferred embodiments of the invention, elements corresponding in their function receive corresponding reference signs even with differing design or formation.

In the figures:

FIG. 1 shows a schematic representation of a CIT endoscope according to the invention as part of a video endoscopy system,

FIG. 2 shows a very schematized view of the stereoscopic imaging optical unit of an endoscope as is previously known in the prior art,

FIG. 3 shows a schematic view of the sensor surface of the image sensor of the endoscope according to FIG. 2,

FIG. 4 shows a very schematized view of a stereoscopic imaging optical unit, which is embodied in the distal end section of an endoscope according to the invention having an optical correction element according to the invention,

FIG. 5 shows a top view of the sensor surface of the image sensor of the endoscope according to the invention according to FIG. 4,

FIG. 6 shows a very simplified representation, which is not to scale, of an optical correction element in the sense of the invention,

FIG. 7 shows a further possible embodiment of an optical correction element according to the invention,

FIG. 8 shows a lateral view of a further optical correction element according to the invention, which can be used in an endoscope according to the invention, which can sensorially capture different spectral ranges separately,

FIG. 9 shows the optical correction element from FIG. 8 in a perspective view,

FIG. 10 shows a schematized side view of the optical elements in the distal end section of an endoscope according to the invention, and finally

FIG. 11 shows a further possible embodiment according to the invention in which the two image circles overlap.

### DETAILED DESCRIPTION

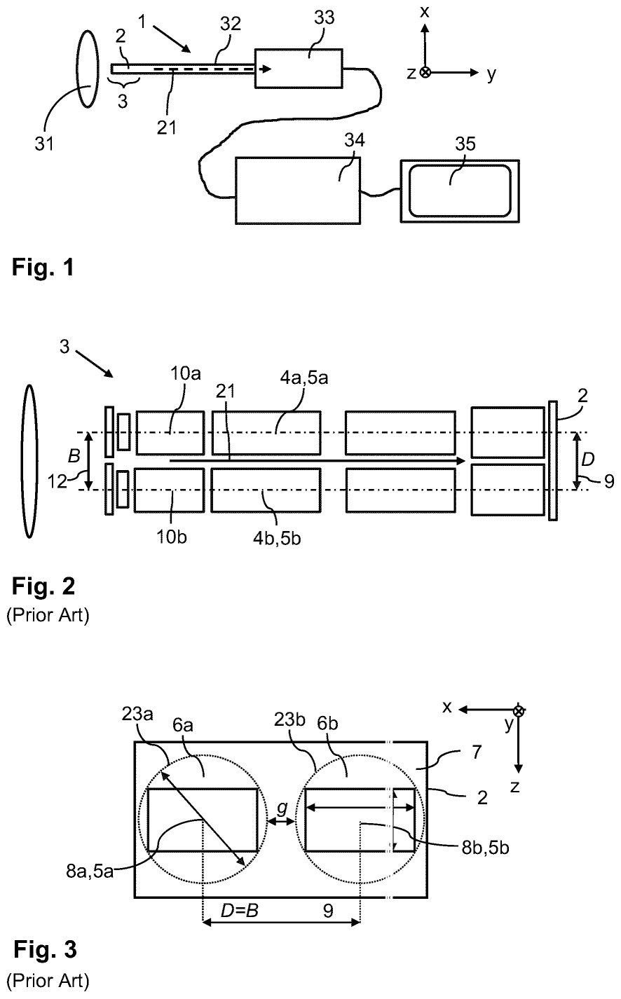

FIG. 1 shows a typical application situation, in which an endoscope 1 according to the invention can be used. The endoscope 1 has an endoscope shaft 32, at the proximal end of which a handle 33 is arranged, using which the operator can position the endoscope. At the distal end of the endoscope shaft 32, an image sensor 2 of the endoscope 1 is arranged in a distal end section 3, so that it is embodied as a so-called chip-in-tip (CIT) endoscope. The image signals generated by the image sensor 2 are transmitted to a camera control unit 34, which converts the signals into a video signal and transmits it to a display unit 35 for display thereon.

As FIG. 2 shows, forming two optical channels 4a and 4b with the aid of multiple optical elements in a CIT endoscope 1 as illustrated in FIG. 1 is already previously known in the prior art. In this way, each of the two optical channels 4a and 4b defines an associated respective imaging beam path 5a, 5b, which both end on a jointly used sensor surface 7 of the image sensor 2 shown in FIG. 2, in order to generate there a respective left or right individual image of the scene which is currently being observed using the endoscope 1.

As a view of FIG. 3 shows, the imaging beam path 5a of the right optical channel 4a generates a right image area 6a used for imaging and in an analogous manner the left imaging beam path 5b generates the illustrated left image area 6b. Since the optical elements of the two channels 4a and 4b are largely designed as rotationally symmetrical, as is typical in the prior art, two respective image circles 23a and 23b are therefore generated by the respective imaging beam path 5a, 5b on the sensor surface 7. The centers of these image circles 23a, 23b simultaneously form respective geometrical image center points 8a and 8b of the two image areas 6a, 6b. In this case, the pixels in the image areas 6a, 6b can be read out to generate respective individual images having a format in the form of a circular disk; or else only pixels in the image subareas 46a, 46b identified by rectangular boxes can also be read out to generate rectangular images.

As the view of FIG. 2 shows, each of the two imaging beam paths 5a and 5b defines the respective optical axis 10a, 10b, which extends through the respective optical channel 4a, 4b, on the entry side. These two optical axes 10a, 10b have a distance B, which defines a stereo base of this endoscope 1, on the entry side. With the aid of the two individual images which are simultaneously sensorially captured by the image sensor 2, stereoscopic images can be obtained, wherein the size of the stereo base B determines how strong the three-dimensional impression is, which the observer obtains upon observing these stereoscopic images. Since the two optical axes 10a and 10b extend strictly in parallel up to the image sensor 2 in the example of FIG. 2, the distance D, at which the two optical axes 10a, 10b are incident on the sensor surface 7, is equal to the stereo base B, so that D=B holds true.

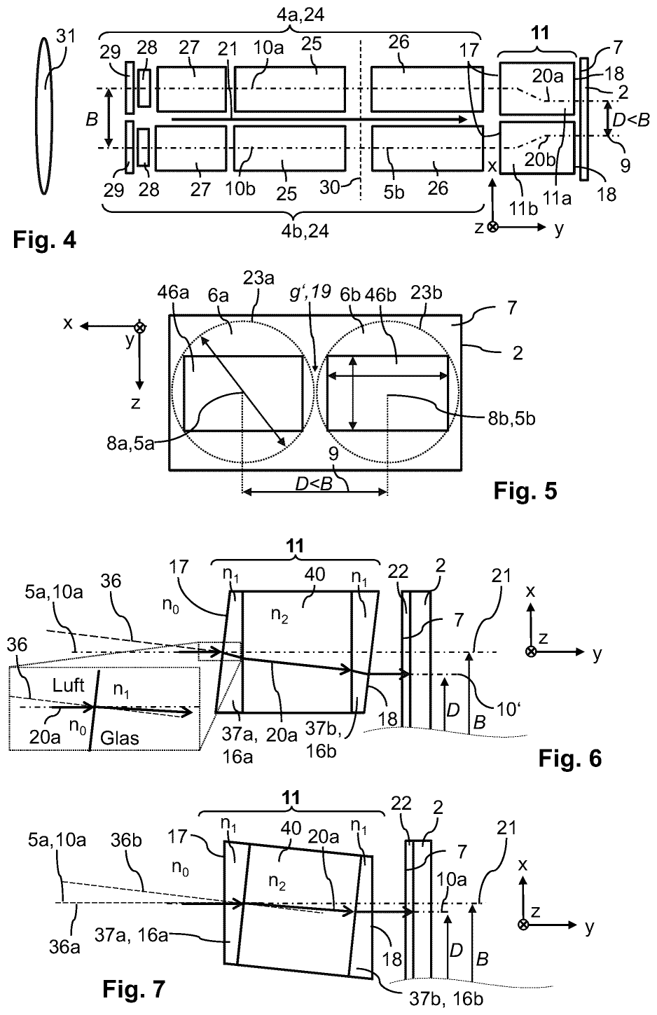

FIG. 4 shows a first example according to the invention of a stereoscopic CIT endoscope 1, wherein the two optical channels 4a, 4b can also be seen here (analogously to FIG. 2), which form a respective imaging beam path 5a, 5b, which both end on the jointly used sensor surface 7 of the image sensor 2, wherein the entire optical arrangement shown in FIG. 4 is arranged in the distal end section 3 of the endoscope 1, as was illustrated in FIG. 1. In comparison to the previously known design of FIG. 2, however, it can be seen in FIG. 4 that an optical correction element 11 is arranged between the two optical channels 4a and 4b and the image sensor 2. This optical correction element 11, which is constructed in two parts from the two correction elements 11a and 11b in the example of FIG. 2, recognizably shifts both optical axes 10a and 10b here in each case axially parallel toward one another so that the resulting above-explained image center distance D is now less than the entry-side stereo base B.

As FIG. 5 shows—at equal sensor size—due to the reduced image center distance D, the two image circles 23a and 23b can now be made larger and approach closer to one another, so that only a negligibly small gap g′ now remains between the two image circles 23a, 23b. Due to the absolutely enlarged image circles 23a, 23b, more pixels can now be read out for the respective individual image, because of which the image resolution increases.

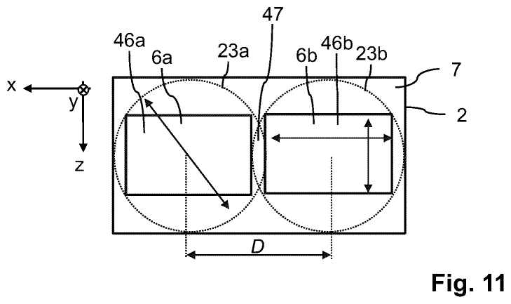

It can also be seen in FIG. 4 that the respective imaging optical unit 24, which forms the respective optical channel 4a, 4b, comprises in each case an objective optical unit 25 and a downstream relay optical unit 26. The respective relay optical unit 26 images the respective intermediate image from the intermediate image plane 30 shown through the optical correction element 11 on the sensor surface 7. It can furthermore also be seen that a deflection prism 27 is arranged before the objective optical unit 25, which receives imaging beams from a concave lens 28 arranged before it. As FIG. 10 illustrates, in this way an oblique view can be formed for both optical channels 4a, 4b of the endoscope 1.

The entire respective imaging optical unit 24 is arranged here before the optical correction element 11 in each case. The imaging light beams emerging from the optical correction element 11 therefore only still have to pass through a cover glass 22 of the image sensor 2 (cf. FIG. 6 and FIG. 7, for example), before they are incident on the sensor surface 7.

FIGS. 6 and 7 show first possible embodiments of an optical correction element 11 according to the invention, specifically as a refractive element in each case. It can be seen in both embodiments of FIGS. 6 and 7 that the optical correction element 11 is embodied by means of two complementarily formed optical deflection elements 37a and 37b. These elements are implemented by identically formed glass wedges 16a and 16b, which are applied to the planar end faces of a glass body 40. The front and the rear glass wedge 16a and 16b cooperate here so that the optical axis 10a or the main beam 20a shown of the illustrated imaging beam path 5a of the right optical channel 4a is shifted in parallel in the negative x direction. Due to mirror-symmetric formation of a further optical correction element 11 in the left optical channel 4b, if necessary, a further additional parallel shift of the main beam 20b there could be generated in each case, but then in the positive x direction, so that a situation as illustrated in FIG. 4 would occur, in which the two main beams 20a, 20b (which extend along the optical axis) approach.

It can be seen in each of the two exemplary embodiment of FIGS. 6 and 7 that the respective associated axis 10a is shifted in parallel after passage through the optical correction element 11, so that the distance to the adjacent optical axis of the adjacent optical channel is necessarily reduced. In both examples of FIGS. 6 and 7, the respective entry surface 17 and the associated exit surface 18 of the optical correction element 11 are moreover aligned parallel to one another. However, the entry surface 17 also extends parallel to the sensor surface 7 of the image sensor 2 only in the example of FIG. 7, while in the example of FIG. 6, the entry surface 17 and also the parallel exit surface 18 are each aligned nonparallel to the sensor surface 7. Therefore, the main beam 20a is also initially not refracted upon entry into the correction element 11 of FIG. 7, but rather only at the internal glass/glass interfaces, which exist between the glass body 40 (having index of refraction n2) and the two glass wedges 16a, 16b (each having index of refraction n1).

In the example of FIG. 6, in contrast, the main beam 20a entering the optical correction element 11 is deflected four times, wherein the left detail view in FIG. 6 shows the first optical refraction at the first air-glass interface, at which the entering main beam 20a is initially diffracted toward the perpendicular of incidence 36. If the course of the main beam 20a in FIG. 6 is followed, a second optical refraction can be seen at the following glass/glass interface, a third optical refraction again at a glass/glass interface, and finally the fourth and last optical refraction upon exiting from the exit surface 18 of the correction element 11.

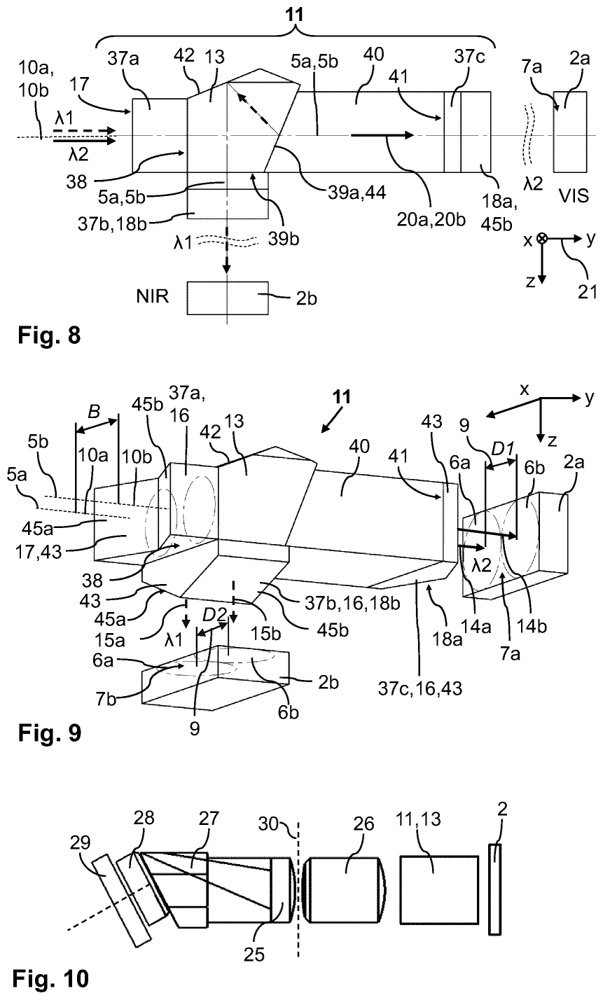

FIG. 8 shows a further particularly advantageous embodiment, in which the optical correction element 11 has an entry surface 17 and two exit surfaces 18a and 18b, which are spatially separated from one another. A wavelength-selective mirror surface 44 can also be seen, which splits the respectively incident main beam 20a/20b in a wavelength-selective manner into a first imaging path 14a/14b and a second imaging path 15a/15b (cf. also the perspective view according to FIG. 9 in this regard). Accordingly, in the example of FIG. 8, a respective image sensor 2a, 2b is assigned to each of the two exit surfaces 18a and 18b. These image sensors 2a, 2b which are spatially separated from one another (their distances from the respective exit surface 18a, 18b are not shown to scale) thus enable imaging in two different wavelength ranges, because visible wavelengths (VIS) pass through the mirror surface 44 and are thus sensorially acquired by the first image sensor 2a. Fluorescence wavelengths in the near infrared (NIR), in contrast, are reflected by the mirror surface 44, then reflected again at a further (fully mirrored) second mirror surface 42, to finally exit from the second exit surface 18b from the optical correction element 11. These fluorescence wavelengths are therefore sensorially acquired by the second image sensor 2b, wherein this applies for both imaging beam paths 5a and 5b, which are generated by the two optical channels 4a, 4b. This is because both imaging beam paths 5a and 5b, after the wavelength-selective splitting, reach both the first image sensor 2a and the second image sensor 2b, as can be seen well in FIG. 9.

With the aid of the wavelength-selective mirror surface 44, the beam splitter 13 shown in FIGS. 8 and 9 therefore separates the imaging beams of the respective imaging beam path 5a or 5b into a right first imaging path 14a and a left first imaging path 14b, which both end in the first image sensor 2a, and into a left second imaging path 15a and a right second imaging path 15b, which both end in the second image sensor 2b.

As in the examples of FIGS. 6 and 7, the correction element 11 is also implemented by means of optical deflection elements 37 in the example of FIGS. 8 and 9. The above-described wavelength-selective beam splitter 13 can thus be seen as part of the optical correction element 11 in FIGS. 8 and 9. A double glass wedge 43 in each case forms the entry surface 17 and the two exit surfaces 18a and 18b, which are spatially separated from one another, for each of the total of four imaging paths 14a, 14b, 15a, and 15b here. The respective double glass wedge 43 has two planar optical surfaces 45a and 45b in each case here, which are inclined in relation to one another. The embodiment of FIGS. 8 and 9 therefore corresponds to that of FIG. 6, because the respective main beam 20 of the respective imaging beam path 5a/5b is also already refracted here upon incidence on the entry surface 17 of the correction element 11.

In consideration of the active principle shown in FIG. 6 and the location of the respectively symmetrically formed double glass wedges 43, it is comprehensible that due to the cooperation of the two glass wedges 43 at the entry surface 17 and the first exit surface 18a, the optical correction element 11 shifts the location of both optical axes of the two first imaging paths 14a and 14b, which end on the first image sensor 2a, so that the associated first image center distance D1 resulting is less than the entry-side stereo base B. This also applies to the second image center distance D2 in the area of the second image sensor 2b, since here the two glass wedges 43 in the area of the entry surface 17 and in the area of the second exit surface 18b cooperate accordingly.

It is also to be mentioned with reference to FIGS. 8 and 9 that, for example, the lower double glass wedge 43 in the area of the second exit surface 18b of the optical correction element 11 could also be replaced by one or two diffractive elements. Alternatively to the use of diffractive or refractive elements, it would also be conceivable, for example, to tilt the upper second mirror surface 42 so that a desired beam deflection is achieved in order to shift the respective main beam in parallel. In this case, the deflection would therefore (at least partially) be implemented using a reflective optical element.

It can furthermore be seen well in FIGS. 8 and 9 that a separate glass body 40 is placed on the wavelength-selective mirror surface 44 of the beam splitter 13, since in this way the optical path length differences can be compensated for. The already mentioned third optical deflection element 37c, which is formed by one of the three double glass wedges 43, is applied in this case to an exit surface 41 of this glass body 40.

The respective entry surfaces 17 and the associated exit surfaces 18 are also each aligned parallel to one another in the example of FIGS. 8 and 9, if the respectively correct surface which is associated with the respective imaging path 14a/14b/15a/15b is observed. Furthermore, it can be seen (as in the example of FIG. 6) that the entry and exit surfaces 17, 18 of the optical correction element 11 are each aligned nonparallel to the respective sensor surface 7a or 7b of the respective image sensor 2a/2b. After passing through the optical correction element 11, the respective main beam 20a, 20b of the two paths 14a, 14b extends strictly along the y direction, however, which corresponds to the longitudinal axis 21 of the endoscope (cf. FIG. 8 and FIG. 1).

FIG. 11 shows a further embodiment according to the invention, which is possible if the mechanical tolerances in the assembly of the endoscope are controlled sufficiently well, and in which the two image circles overlap in the illustrated overlap area 47 on the sensor surface 7. An additional gain in image resolution can be achieved by such an embodiment, because the rectangular, nonoverlapping image subareas 46a, 46b (which can still be read out to generate rectangular images) on the image sensor surface 7 result as even larger in comparison to the example of FIG. 5; the resulting proportion of these rectangular image subareas 46a, 46b on the sensor surface 7 is also greater, so that more pixels are available for the respective rectangular individual image. However, in such an embodiment, adequate measures are to be taken to minimize scattered light, in order to avoid optical crosstalk between the areas 46a and 46b. Furthermore, it can be seen in FIG. 11 that the two image subareas 46a, 46b are still spaced apart from one another for this reason (namely advantageously at least by the x width of the overlap area 47), thus just do not overlap.

In summary, to improve the image quality of stereoscopic images which are recorded using a CIT endoscope 1, it is provided that an optical correction element 11, which shifts respective main beams 20a, 20b of the respective optical channel 4a, 4b axially parallel so that imaging beam paths 5a and 5b, which are generated by the two optical channels 4a and 4b, approach one another, is arranged between two optical channels 4a, 4b which are used for imaging, extend parallel and are embodied identically, and the image sensor 2 used for imaging. As a result, an image center distance D between image areas 6a and 6b on a sensor surface 7 of the image sensor 2 that is used for both individual images can thus be reduced in comparison to an entry-side stereo base B defined by the two optical channels 4a and 4b, so that a higher image resolution can be achieved (cf. FIG. 9).

### LIST OF REFERENCE SIGNS

1 endoscope
2 image sensor (arranged in 3)
3 distal end section (of 1)
4 optical channel (formed by an imaging optical unit)
5 imaging beam path (extends through/is defined by 4)
6 image area (on 7)
7 sensor surface (of 2)
8 geometrical image center point (of 6)
9 image center distance
10 optical axis (defined center of 5)
11 optical correction element
12 stereo base B
13 beam splitter (splits 5 into two optical paths)
14 first imaging path (for example used for white light imaging (VIS))
15 second imaging path (for example used for fluorescent light imaging (FI))
16 glass wedge
17 entry surface (of 11)
18 exit surface (of 11)
19 safety area
20 main beam (of 5)
21 longitudinal axis (of 1)
22 cover glass (protects 7 from dust and mechanical stress)
23 image circle
24 imaging optical unit
25 objective optical unit
26 relay optical unit
27 deflection prism
28 concave lens
29 entry window
30 intermediate image plane
31 tissue (observed using 1)
32 endoscope shaft
33 handle
34 camera control unit (CCU)
35 display unit
36 perpendicular of incidence
37 optical deflection element
38 entry surface (of 13)
39 exit surface (of 13)
40 glass body
41 exit surface (of 40)
42 mirror surface (of 13; can be made fully mirrored)
43 symmetrical double glass wedge
44 wavelength-selective mirror surface (of 13)
45 planar optical surface
46 rectangular image subarea
47 overlap area (of 6a and 6b on 7)

1. A stereoscopic chip-in-tip endoscope (1), comprising:
at least one distal image sensor (2), which is arranged in a distal end section (3) of the endoscope (1), the at least one image sensor (2) including a sensor surface (7);
two optical channels (4a, 4b) having respective imaging beam path (5a, 5b), which generate a right image area (6a) used for imaging and a corresponding left image area (6b) on the sensor surface (7);
respective geometrical image center points (8a, 8b) of the right and left image areas (6a, 6b) define an image center distance D;
each of the two imaging beam paths (5a, 5b) defines a respective optical axis (10a, 10b) on an entry side, which extends through the respective optical channel (4a, 4b);
the two optical axes (10a, 10b) are arranged on the entry side at a distance B, which defines a stereo base (12) of the endoscope (1);
wherein the endoscope (1) comprises an optical correction element (11), which is arranged between the two optical channels (4a, 4b) and the at least one image sensor (2) and which is configured to shift at least one of the two optical axes (10a, 10b) so that D<B.

2. The endoscope (1) as claimed in claim 1, wherein the optical correction element (11) is configured to shift at least one of the two optical axes (10a, 10b) axially parallel.

3. The endoscope (1) as claimed in claim 1, wherein a respective entire imaging optical unit (24) of the respective optical channel (4a, 4b) is arranged before the optical correction element (11) in a direction of the respective imaging beam path (5a, 5b).

4. The endoscope (1) as claimed in claim 1, wherein the optical correction element (11) has an entry surface (17) and two exit surfaces (18a, 18b), which are spatially separated from one another.

5. The endoscope (1) as claimed in claim 4, wherein the correction element (11) furthermore has a wavelength-selective mirror surface (44), which is configured to guide wavelengths in a first wavelength range onto a first exit surface (18a) of the correction element (11) and wavelengths in a second wavelength range, deviating from the first, onto a second exit surface (18b) of the correction element (11);
each of the entry or first and second exit surfaces (17, 18a, 18b) is formed by a respective double glass wedge (43), which forms two optical surfaces (45a, 45b), which are inclined in relation to one another and are each planar; and
the respective imaging beam path (5a, 5b) extends through only one of the two optical surfaces (45a, 45b) in each case.

6. The endoscope (1) as claimed in claim 1, wherein
the optical correction element (11) comprises a wavelength-selective beam splitter (13).

7. The endoscope (1) as claimed in claim 4, wherein the at least one image sensor comprises two spatially separated image sensors (2a, 2b) that are assigned to the beam splitter (13), so that respective imaging in two different wavelength ranges is enabled using the two image sensors (2a, 2b); and
the beam splitter (13) is configured to spatially separate imaging beams of the respective imaging beam path (5a, 5b) into
a right first imaging path (14b) and a left first imaging path (14b) and into
a right second imaging path (15b) and a left second imaging path (15b).

8. The endoscope (1) as claimed in claim 7, wherein
the optical correction element (11) is configured to shift both the location of at least one optical axis of the two first imaging paths (14a, 14b) and the location of at least one optical axis of the two second imaging paths (15a, 15b), such that both a first image center distance D1 associated with the first imaging paths (14a, 14b) and a second image center distance D2 associated with the second imaging paths (15a, 15b) is adapted.

9. The endoscope (1) as claimed in claim 1, wherein
each of the two optical channels (4a, 4b) has a respective imaging optical unit (24), which comprises an objective optical unit (25) and a relay optical unit (26).

10. The endoscope (1) as claimed in claim 9, wherein
the relay optical unit (26) is configured to image an image from an intermediate image plane (30), which is generated by the objective optical unit (25), through the optical correction element (11) on the sensor surface (7) of the at least one image sensor (2); and
the respective imaging optical unit (24) additionally comprises a deflection prism (27) arranged before the objective optical unit (25) and a concave lens (28) arranged before the deflection prism (27), so that the endoscope (1) is embodied having an oblique view.

11. The endoscope (1) as claimed in claim 1, wherein
the optical correction element (11) is embodied by at least two optical deflection elements (37a, 37b).

12. The endoscope (1) as claimed in claim 11, wherein the at least two optical deflection elements (37a, 37b) comprise at least two complementary glass wedges (16a, 16b), and the two optical deflection elements (37a, 37b) cooperate so that the respective associated optical axis (10a, 10b) is shifted in parallel after passage through the optical correction element (11), so that the two optical axes (10a, 10b) extend at a reduced distance and the left and the right image area (6a, 6b) approach one another on the sensor surface (7).

13. The endoscope (1) as claimed in claim 11, wherein the optical correction element (11) comprises a wavelength-selective beam splitter (13), and the at least two optical deflection elements (37a, 37b) are applied to an entry surface (38) and to an exit surface (39) of the beam splitter (13), and
a third optical deflection element (37c) is applied to an exit surface (41) of a glass body (40), which is placed on a wavelength-selective mirror surface (44) of the beam splitter (13), which wavelength-selective mirror surface (44) forms a first exit surface (39b) of the beam splitter (13).

14. The endoscope (1) as claimed in claim 1, wherein
an entry surface (17) and an associated exit surface (18) of the optical correction element (11) are aligned parallel to one another.

15. The endoscope (1) as claimed in claim 14, wherein
the entry surface (17) and the exit surface (18) of the optical correction element (11) extend parallel or nonparallel to the sensor surface (7) of the at least one image sensor (2) and/or
a respective main beam (20a, 20b) is adapted to extend obliquely in relation to a longitudinal axis (21) of the endoscope (1) during the passage through the optical correction element (11) and parallel to the longitudinal axis (21) of the endoscope (1) after exit from the optical correction element (11).

16. The endoscope (1) as claimed in claim 1, wherein
a respective main beam (20a, 20b) is configured to be deflected at least four times during the passage through the optical correction element (11) and/or
the respective main beam (20a, 20b), during the passage through the optical correction element (11), initially is adapted to experience
a first optical refraction at an air/glass interface, then
a second optical refraction at a glass/glass interface, then
a third optical refraction at a glass/glass interface, and finally
a fourth and last optical refraction at a glass/air interface, by which the respective main beam (20a, 20b) is adapted to be shifted axially parallel overall.

17. The endoscope (1) as claimed in claim 1, wherein a respective main beam (20a, 20b) is adapted to be refracted twice at an internal glass/glass interface during the passage through the optical correction element (11) and an entry surface (17) and an exit surface (18) of the optical correction element (11) extend parallel to the sensor surface (7) of the at least one image sensor (2).

18. The endoscope (1) as claimed in claim 1, wherein a ratio of the stereo base B and the image center distance D (9) is:
D/B<0.95, preferably it holds true that: D/B<0.90.

19. The endoscope (1) as claimed in claim 1, wherein a deflection of at least one of the two optical axes (10a, 10b) caused by the optical correction element (11) is designed such that the two image areas (6a, 6b) do not overlap on the sensor surface (7), and a safety area (19) having a width of at least 50 μm remains between the two image areas (6a, 7b), which is not used for imaging.

20. The endoscope (1) as claimed in claim 1, wherein a deflection of at least one of the two optical axes (10a, 10b) that is adapted to be caused by the optical correction element (11) is designed such that the two image areas (6a, 6b) overlap in an overlap area (47) on the sensor surface (7), and
only smaller image subareas (46a, 46b) within the two image areas (6a, 6b) are used in each case for imaging.
